(19) United States
(12) Patent Application Publication
(10) Pub. No.: US 2025/0032451 A1
(43) Pub. Date: Jan. 30, 2025

(54) MICROSPHERE FORMULATIONS COMPRISING ASENAPINE AND METHODS FOR MAKING AND USING THE SAME

(71) Applicant: Oakwood Laboratories, LLC, Oakwood Village, OH (US)

(72) Inventors: Emma Murphy, Kent, OH (US); Brad Minrovic, Willoughby, OH (US); Matthew Cornell, Mayfield Heights, OH (US); Eli McCarty, Oakwood Village, OH (US); Tracy Richey, Kent, OH (US); Mark Smith, Oakwood Village, OH (US); Nick DeLucia, Oakwood Village, OH (US)

(21) Appl. No.: 18/713,417

(22) PCT Filed: Nov. 22, 2022
(86) PCT No.: PCT/US22/80292

§ 371 Date: May 24, 2024

Related U.S. Application Data
(60) Provisional application No. 63/264,544, filed on Nov. 24, 2021.
(60) Provisional application No. 63/267,405, filed on Feb. 1, 2022.

(51) Int. Cl. A61K 31/407; A61K 9/16

(52) U.S. Cl. CPC A61K 31/407; A61K 9/1647

(57) ABSTRACT

Microsphere formulations comprising asenapine are provided. Methods for making and using the microsphere formulations are also provided.

### CROSS-REFERENCE TO RELATED APPLICATIONS

This application claims priority from U.S. Provisional Patent Application No. 63/267,405, filed on Feb. 1, 2022, and U.S. Provisional Patent Application No. 63/264,544, filed on Nov. 24, 2021, each of which is incorporated by reference herein in its entirety.

### BACKGROUND

Asenapine (chemical formula C₁₇H₁₆ClNO; CAS Number 65576-45-6; IUPAC name: (3aRS, 12bRS)-rel-5-Chloro-2,3,3a, 12b-tetrahydro-2-methyl-1H-dibenz [2,3:6,7]oxepino [4,5-c]pyrrole), characterized by the structure:

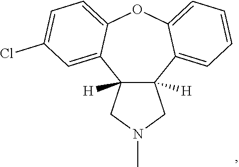 [SMILES: CN1C[C@@H]2c3ccccc3Oc3ccc(Cl)cc3[C@H]2C1]

is an atypical antipsychotic medication used to treat schizophrenia and acute mania associated with bipolar disorder. Approved as a generic medication in the United States in 2020, asenapine is currently available under the brand names Saphris® as a twice-daily orally disintegrating sublingual tablet and Secuado® as a once-daily transdermal administration.

A need exists for an extended-release asenapine-encapsulating microsphere formulation, especially one having a high drug load (>˜15% by weight), small particle size (about 15-40 μm average diameter (D₅₀)), and long release duration (˜30 days).

### SUMMARY

Microsphere formulations comprising asenapine are provided. The microsphere formulations comprise polymer microspheres, each polymer microsphere comprising: (i) asenapine; and (ii) a biodegradable polymer comprising a polylactide polymer (a “PLA”), wherein each polymer microsphere comprises a drug load of asenapine of at least about 15% by weight of the polymer microsphere, and wherein the polymer microspheres have an average particle size of about 15 μm to about 40 μm. In one aspect, the microsphere formulations are characterized in that the asenapine is released over a period of about 30 days.

In one aspect, the microsphere formulations may be made by a method, the method comprising: (A) mixing: (i) the biodegradable polymer comprising a PLA; (ii) a primary solvent; (iii) asenapine; and (iv) a co-solvent, to form a dispersed phase; (B) mixing: (i) water; and (ii) a surfactant, to form a continuous phase; and (C) combining the dispersed phase with the continuous phase in a homogenizer.

In one aspect, a method for treating schizophrenia and acute mania associated with bipolar disorder is provided. The method may comprise administering by intramuscular or subcutaneous injection to a patient in need thereof a microsphere formulation made according to the methods described herein, wherein the formulation is administered to the patient with a dosing schedule of about every 30 days.

In another aspect, use is disclosed of a microsphere formulation comprising polymer microspheres, each polymer microsphere comprising: (i) asenapine; and (ii) a biodegradable polymer comprising a PLA, wherein each polymer microsphere comprises a drug load of asenapine of at least about 15% by weight of the polymer microsphere, and wherein the polymer microspheres have an average particle size of about 15 μm to about 40 μm (D₅₀), in the manufacture of a medicament for the treatment of schizophrenia and acute mania associated with bipolar disorder.

In another aspect, a microsphere formulation comprising polymer microspheres, each polymer microsphere comprising: (i) asenapine; and (ii) a biodegradable polymer comprising a PLA, wherein each polymer microsphere comprises a drug load of asenapine of at least about 15% by weight of the polymer microsphere, and wherein the polymer microspheres have an average particle size of about 15 μm to about 40 μm (D₅₀), is provided for use as a medicament for the treatment of schizophrenia and acute mania associated with bipolar disorder.

In another aspect, a kit is provided, the kit comprising polymer microspheres, each polymer microsphere comprising: (i) asenapine; and (ii) a biodegradable polymer comprising a PLA, wherein each polymer microsphere comprises a drug load of asenapine of at least about 15% by weight of the polymer microsphere, and wherein the polymer microspheres have an average particle size of about 15 μm to about 40 μm (D₅₀).

### BRIEF DESCRIPTION OF THE FIGURES

FIG. 1 is a schematic depicting a method for making asenapine-encapsulated polymer microspheres.

FIG. 2 is a graph showing an amount of asenapine released in vitro over time from several example asenapine-encapsulated polymer microspheres.

FIG. 3 is a graph showing the measured mean blood concentration (ng/ml) of asenapine as a function of time in a pharmacokinetics study in rats using several example asenapine-encapsulated polymer microspheres.

FIG. 4 is a graph showing the measured mean blood concentration (ng/ml) of asenapine as a function of time in a pharmacokinetics study in rats using several example asenapine-encapsulated polymer microspheres.

### DETAILED DESCRIPTION

Microsphere formulations comprising asenapine are provided. The microsphere formulations comprise polymer microspheres, each polymer microsphere comprising: (i) asenapine; and (ii) a biodegradable polymer comprising a PLA, wherein each polymer microsphere comprises a drug load of asenapine of at least about 15% by weight of the polymer microsphere, and wherein the polymer microspheres have an average particle size of about 15 μm to about 40 μm. In one aspect, the microsphere formulations are characterized in that the asenapine is released over a period of about 30 days.

In one aspect, the microsphere formulations may be made by a method, the method comprising: (A) mixing: (i) the biodegradable polymer comprising a PLA; (ii) a primary solvent; (iii) asenapine; and (iv) a co-solvent, to form a dispersed phase; (B) mixing: (i) water; and (ii) a surfactant, to form a continuous phase; and (C) combining the dispersed phase with the continuous phase in a homogenizer.

### Asenapine

In one aspect, the asenapine is a free base. In one aspect, the asenapine is a salt. In one aspect, the asenapine salt is asenapine maleate supplied by Olon S.p.A. In one aspect, the asenapine maleate has a water solubility of 3 mg/mL, a dichloromethane (DCM) solubility of 324 mg/mL, a benzyl alcohol (BA) solubility of 500 mg/mL, an ethyl acetate (EA) solubility of 10 mg/mL, a dimethyl sulfoxide (DMSO) solubility of 700 mg/mL, an N-methylpyrrolidone (NMP) solubility of 700 mg/mL, and an ethanol (EtOH) solubility of 100 mg/mL. In one aspect, the asenapine maleate has a pKa=8.64.

### Biodegradable Polymers

In one aspect, the biodegradable polymer is a PLA. The PLA may have an inherent viscosity of about 0.1 dL/g to about 0.35 dL/g, including from about 0.13 dL/g to about 0.29 dL/g, and from about 0.25 dL/g to about 0.35 dL/g, including 0.13 dL/g, 0.14 dL/g, 0.16 dL/g, 0.18 dL/g, and 0.29 dL/g. In one aspect, the PLA is acid terminated. In one aspect, the PLA comprises Viatel® DL 02 A, acid terminated, IV=0.13 dL/g, 0.14 dL/g, 0.16 dL/g, or 0.18 dL/g, supplied by Ashland. In one aspect, the PLA comprises Resomer® R 203 H, acid terminated, IV=0.29 dL/g, supplied by Evonik.

### Dispersed Phase

In one aspect, the dispersed phase comprises a primary solvent. In one aspect, the primary solvent comprises DCM. The dispersed phase may also include up to about 50% by weight of a co-solvent capable of optimizing the solubility of asenapine. In one aspect, the co-solvent may be BA, DCM, DMSO, dimethyl formamide (DMF), dimethyl acetamide (DMAc), acetonitrile(ACN), EtOH, NMP, EA, or any other solvent that increases the solubility of asenapine in the dispersed phase containing DCM. In one aspect, the primary solvent comprises DCM, and the co-solvent comprises BA. In one aspect, the ratio of DCM to BA is about 3:1. The organic solvent is removed from the microspheres in the course of their preparation. A microsphere is considered to be “essentially free” of organic solvent if the microsphere meets the standards set forth in the “ICH Harmonised Guideline, Impurities: Guideline for Residual Solvents Q3C(R8), Current Step 4 version dated 22 Apr. 2021,” which is incorporated herein by reference in its entirety.

### Continuous Phase

The dispersed phase may be combined with an aqueous continuous phase that comprises water and, optionally, a surfactant. The surfactant component may be present in the continuous phase in an amount of about 0.35% to about 1.0% by weight in water. In one aspect, the surfactant component comprises polyvinyl alcohol (PVA) in a concentration of about 0.35% by weight in water. In one aspect, the surfactant component comprises PVA in a concentration of about 1% by weight in water.

In some aspects, the dispersed phase flow rate to the homogenizer may be from about 10 mL/min to about 30 mL/min, including about 20 mL/min and about 25 mL/min. In some aspects, the continuous phase flow rate to the homogenizer may be about 2 L/min. Thus, in one aspect, the continuous phase: dispersed phase ratio may be from about 66:1 to about 200:1, including about 100:1 and about 80:1.

The continuous phase may be provided at room temperature or above or below room temperature. In some aspects, the continuous phase may be provided at about 40° C., about 37° C., about 35° C., about 30° C., about 25° C., about 20° C., about 15° C., about 10° C., about 5° C., about 0° C., and any range or value between any of those values.

### Homogenizer

For brevity, and because the methods are equally applicable to either, the phrase “homogenizer” contemplates a system or apparatus that can homogenize the dispersed phase and the continuous phase, emulsify the dispersed phase and the continuous phase, or both, which systems and apparatuses are known in the art. For example, in one aspect, the homogenizer is an in-line Silverson Homogenizer (commercially available from Silverson Machines, Waterside UK) or a Levitronix® BPS-i100 integrated pump system used, e.g., as described in U.S. Pat. No. 11,167,256, which is incorporated by reference herein in its entirety. In one aspect, the homogenizer is a membrane emulsifier. In one aspect, the homogenizer runs at an impeller speed of about 1,000 to about 4,000 revolutions per minute (“RPM”), including about 2,000 RPM and about 2,500 RPM.

### Drug Load

The drug load of each polymer microsphere in a drug to polymer ratio, expressed as a percentage, may be greater than 15 wt/wt %, and from about 15 wt/wt % to about 55 wt/wt %, from about 15 wt/wt % to about 25 wt/wt %, about 20 wt/wt %, about 25 wt/wt %, about 30 wt/wt %, about 35 wt/wt %, about 40 wt/wt %, about 45 wt/wt %, and about 50 wt/wt %, and any range between any two of those values.

### Particle Size

In one aspect, the polymer microspheres may have an average particle size between about 15 μm (D₅₀) and about 40 μm (D₅₀), including between about 15 μm (D₅₀) and about 25 μm (D₅₀), and about 20 μm (D₅₀), about 25 μm (D₅₀), about 30 μm (D₅₀), about 35 μm (D₅₀), and about 40 μm (D₅₀), and any range between any two of those values.

### Extended Release

Where the polymer is a PLA, the microsphere formulations are characterized in that they have a duration of release of at least about two weeks and up to about six weeks. In some aspects, the microsphere formulations have a duration of release of about three weeks, about four weeks, or about five weeks, and any range between any two of those values. In some aspects, the duration of release is about 30 days.

### Therapeutic Benefits

In one aspect, a method for treating schizophrenia and acute mania associated with bipolar disorder is provided. The method may comprise administering by intramuscular or subcutaneous injection to a patient in need thereof a microsphere formulation made according to the methods described herein, wherein the formulation is administered to the patient with a dosing schedule of about every 30 days.

In another aspect, use is disclosed of a microsphere formulation comprising polymer microspheres, each polymer microsphere comprising: (i) asenapine; and (ii) a biodegradable polymer comprising a PLA, wherein each polymer microsphere comprises a drug load of asenapine of at least about 15% by weight of the polymer microsphere, and wherein the polymer microspheres have an average particle size of about 15 μm to about 40 μm (D₅₀), in the manufacture of a medicament for the treatment of schizophrenia and acute mania associated with bipolar disorder.

In another aspect, a microsphere formulation comprising polymer microspheres, each polymer microsphere comprising: (i) asenapine; and (ii) a biodegradable polymer comprising a PLA, wherein each polymer microsphere comprises a drug load of asenapine of at least about 15% by weight of the polymer microsphere, and wherein the polymer microspheres have an average particle size of about 15 μm to about 40 μm (D₅₀), is provided for use as a medicament for the treatment of schizophrenia and acute mania associated with bipolar disorder.

In another aspect, a kit is provided, the kit comprising polymer microspheres, each polymer microsphere comprising: (i) asenapine; and (ii) a biodegradable polymer comprising a PLA, wherein each polymer microsphere comprises a drug load of asenapine of at least about 15% by weight of the polymer microsphere, and wherein the polymer microspheres have an average particle size of about 15 μm to about 40 μm (D₅₀).

### Examples

### Example 1—General Preparation of Polymer Microspheres Comprising Asenapine Via a Single Emulsion Method

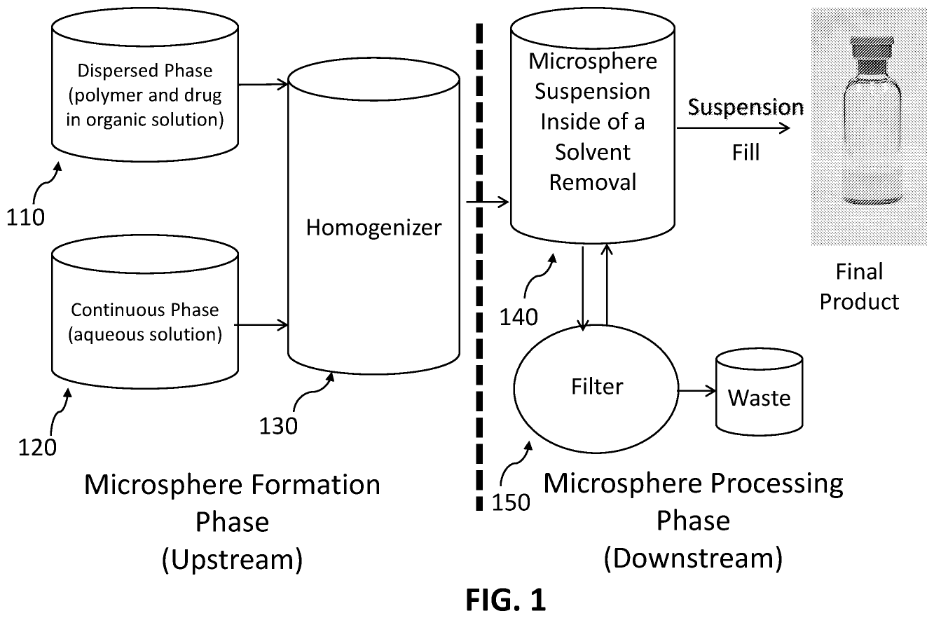

Microsphere Formation Phase. With reference to FIG. 1, a dispersed phase (“DP”) 10 is formed by dissolving a polymer matrix (such as a PLA polymer) in an organic solvent system (such as DMC and BA), followed by the addition of asenapine with mixing until completely dissolved. The DP 10 is filtered using a 0.2 μm sterilizing PTFE or PVDF membrane filter (such as EMFLON, commercially available from Pall or SartoriousAG) and pumped into a homogenizer 30, such as an in-line Silverson Homogenizer (commercially available from Silverson Machines, Waterside UK) or a Levitronix i100 (as described in U.S. Pat. No. 11,167,256), at a defined flow rate. A continuous phase (“CP”) 20 comprising water and surfactant is also pumped into the homogenizer 30 at a defined flow rate. The speed of the homogenizer 30 is generally fixed to achieve a desired polymer microsphere size distribution. A representative continuous “upstream” microsphere formation phase is described in U.S. Pat. No. 5,945,126, which is incorporated by reference herein in its entirety.

Microsphere Processing Phase. The formed or forming microspheres exit the homogenizer 30 and enter a solvent removal vessel (“SRV”) 40. Water may be added to the SRV 40 during microsphere formation to minimize the solvent level in the aqueous medium. After the DP 10 has been exhausted, the CP 20 and water flow rates are stopped, and the washing steps are initiated. Solvent removal is achieved using water washing and a hollow fiber filter (commercially available as HFF from GE Healthcare) 50. A representative “downstream” microsphere processing phase is described in U.S. Pat. No. 6,270,802, which is incorporated by reference herein in its entirety.

The washed microspheres are collected and freeze-dried overnight in a lyophilizer (Virtis) to remove any moisture. The resulting microspheres are a free-flowing off-white bulk powder.

### Example 2-Preparation of Asenapine-Encapsulated PLA Polymer Microspheres-Batches 1-3

Following the general procedure described in Example 1, illustrated in FIG. 1, and detailed in Table 1, the DP was formed by dissolving either 2.25 g (Batches 1 and 2) or 4.50 g (Batch 3) of acid-terminated Viatel® DL 02 A polymer (IV=0.18 dL/g) in either 12.29 g (Batches 1 and 2) or 24.57 g (Batch 3) of DCM and either 4.09 g (Batches 1 and 2) or 8.18 g (Batch 3) of BA (DCM/BA ratio=3:1 vol/vol)), followed by addition of either 3.87 g (Batches 1 and 2) or 7.75 g (Batch 3) of asenapine maleate with mixing until completely dissolved. The DP was filtered and pumped at a flow rate of 25 mL/min into a Levitronix® BPS-i100 integrated pump system operating at 2,000 RPM. The CP comprising 0.35% PVA (Batch 1) or 1.0% PVA (Batches 2 and 3) was also pumped into the homogenizer at a flow rate of 2 L/min (CP:DP=80:1).

The formed or forming microspheres exited the homogenizer and entered the SRV. Room temperature deionized water was added to the SRV (12 volume exchanges). Solvent removal was achieved using water washing (35-39° C.) and a hollow fiber filter. The bulk suspension was collected via filtration and lyophilized to obtain a free-flowing powder.

The process parameters and the characterization data for Batches 1-3 are shown in Table 1:

TABLE 1

| Batch # | 1 | 2 | 3 |
|---|---|---|---|
| Polymer | Viatel ® DL 02 A | Viatel ® DL 02 A | Viatel ® DL 02 A |
| | acid-terminated | acid-terminated | acid-terminated |
| Polymer IV | IV = 0.18 dL/g | IV = 0.18 dL/g | IV = 0.18 dL/g |
| Solvent System | DCM/BA (3:1) | DCM/BA (3:1) | DCM/BA (3:1) |
| Surfactant | PVA 0.35% | PVA 1.0% | PVA 1.0% |
| Homogenizer RPM | 2,000 | 2,000 | 2,000 |
| Total Yield (%) | 40 | 37 | 48 |
| Drug Load (%) | 17.2 | 18.9 | 16.3 |
| Encapsulation | 31.3 | 34.4 | 29.6 |
| Efficiency (%) | | | |
| Residual Solvents (% wt.) | DCM/BA (ND/3.8) | DCM/BA (ND/0.8) | DCM/BA (ND/0.7) |
| Particle Size (D₁₀) | 12 | 10 | 8 |
| Particle Size (D₅₀) | 23 | 20 | 18 |
| Particle Size (D₉₀) | 42 | 34 | 32 |
| Polymer MW (kDa) | 15.8 | 15.9 | 15.8 |
| Microsphere MW | 16.0 | 15.8 | 15.1 |
| (kDa) | | | |

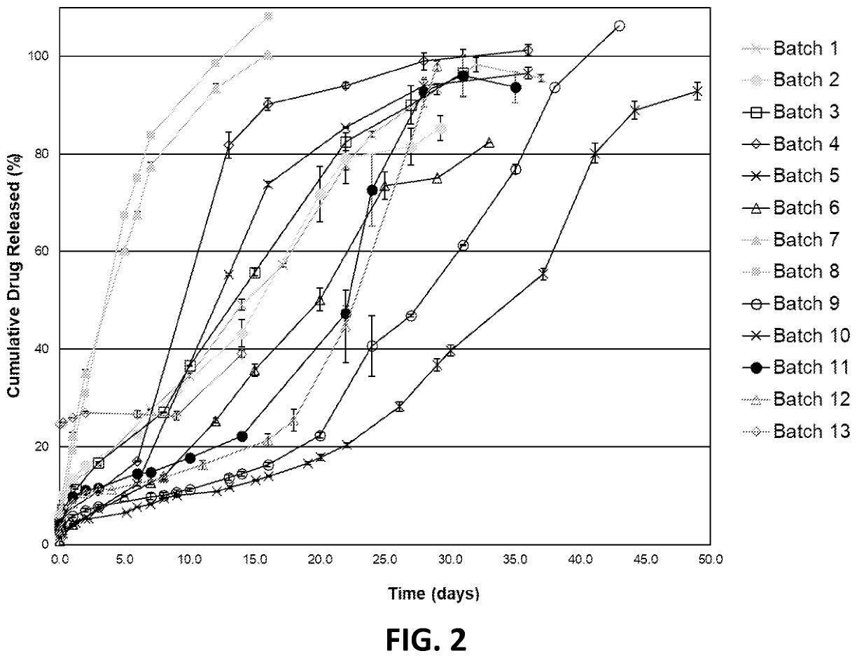

FIG. 2 is a graph showing an amount of asenapine released in vitro over time from asenapine-encapsulated polymer microsphere Batches 1-3.

### Example 3-Preparation of Asenapine-Encapsulated PLA Polymer Microspheres-Batches 4-6

Following the general procedure described in Example 1, illustrated in FIG. 1, and detailed in Table 2, the DP was formed by dissolving either 4.50 g (Batch 4), 5.50 g (Batch 5), or 7.00 g (Batch 6) of acid-terminated Viatel® DL 02 A polymer (IV=0.16 dL/g) in either 24.57 g (Batches 4 and 5) or 24.60 g (Batch 6) of DCM and either 8.18 g (Batches 4 and 5) or 8.20 g (Batch 6) of BA (DCM/BA ratio=3:1 vol/vol)), followed by addition of either 7.75 g (Batch 4), 5.63 g (Batch 5), or 4.23 g (Batch 6) of asenapine maleate with mixing until completely dissolved. The DP was filtered and pumped at a flow rate of 25 mL/min into a Levitronix® BPS-i100 integrated pump system operating at 2,000 RPM. The CP comprising 1.0% PVA was also pumped into the homogenizer at a flow rate of 2 L/min (CP:DP=80:1).

The formed or forming microspheres exited the homogenizer and entered the SRV. Room temperature deionized water was added to the SRV (12 volume exchanges). Solvent removal was achieved using water washing (35-39° C.) and a hollow fiber filter. The bulk suspension was collected via filtration and lyophilized to obtain a free-flowing powder.

The process parameters and the characterization data for Batches 4-6 are shown in Table 2:

TABLE 2

| Batch # | 4 | 5 | 6 |
|---|---|---|---|
| Polymer | Viatel ® DL 02 A | Viatel ® DL 02 A | Viatel ® DL 02 A |
| | acid-terminated | acid-terminated | acid-terminated |
| Polymer IV | IV = 0.16 dL/g | IV = 0.16 dL/g | IV = 0.16 dL/g |
| Solvent System | DCM/BA (3:1) | DCM/BA (3:1) | DCM/BA (3:1) |
| Surfactant | PVA 1.0% | PVA 1.0% | PVA 1.0% |
| Homogenizer RPM | 2,000 | 2,000 | 2,000 |
| Total Yield (%) | 58 | 67 | 66 |
| Drug Load (%) | 27.8 | 21.7 | 14.8 |
| Encapsulation | 51 | 52 | 49 |
| Efficiency (%) | | | |
| Residual Solvents (% wt.) | DCM/BA (ND/0.8) | DCM/BA (ND/1.1) | DCM/BA (ND/1.1) |
| Particle Size (D₁₀) | 9 | 7 | 8 |
| Particle Size (D₅₀) | 18 | 19 | 23 |
| Particle Size (D₉₀) | 31 | 35 | 41 |
| Polymer MW (kDa) | 11.1 | 11.1 | 11.4 |
| Microsphere MW | 11.1 | 11.6 | 11.5 |
| (kDa) | | | |

FIG. 2 is a graph showing an amount of asenapine released in vitro over time from asenapine-encapsulated polymer microsphere Batches 4-6.

### Example 4-Preparation of Asenapine-Encapsulated PLA Polymer Microspheres-Batches 7-8

Following the general procedure described in Example 1, illustrated in FIG. 1, and detailed in Table 3, the DP was formed by dissolving 5.50 g of either acid-terminated Viatel® DL 02 A polymer (IV=0.14 dL/g) (Batch 7) or acid-terminated Viatel® DL 02 A polymer (IV=0.13 dL/g) (Batch 8) in 24.57 g of DCM and 8.18 g of BA (DCM/BA ratio=3:1 vol/vol)), followed by addition of 5.63 g of asenapine maleate with mixing until completely dissolved. The DP was filtered and pumped at a flow rate of 25 mL/min into a Levitronix® BPS-i100 integrated pump system operating at 2,000 RPM. The CP comprising 1.0% PVA was also pumped into the homogenizer at a flow rate of 2 L/min (CP:DP=80:1).

The formed or forming microspheres exited the homogenizer and entered the SRV. Room temperature deionized water was added to the SRV (12 volume exchanges). Solvent removal was achieved using water washing (35-39° C.) and a hollow fiber filter. The bulk suspension was collected via filtration and lyophilized to obtain a free-flowing powder. The process parameters and the characterization data for Batches 7-8 are shown in Table 3:

TABLE 3

| Batch # | 7 | 8 |
|---|---|---|
| Polymer | Viatel ® DL 02 A | Viatel ® DL 02 A |
| | acid-terminated | acid-terminated |
| Polymer IV | IV = 0.14 dL/g | IV = 0.13 dL/g |
| Solvent System | DCM/BA (3:1) | DCM/BA (3:1) |
| Surfactant | PVA 1.0% | PVA 1.0% |
| Homogenizer RPM | 2,000 | 2,000 |
| Total Yield (%) | 60 | 52 |
| Drug Load (%) | 20.4 | 20.6 |
| Encapsulation | 49 | 49 |
| Efficiency (%) | | |
| Residual Solvents (% wt.) | DCM/BA (ND/0.3) | DCM/BA (ND/0.2) |
| Particle Size (D₁₀) | 6 | 7 |
| Particle Size (D₅₀) | 20 | 20 |
| Particle Size (D₉₀) | 38 | 36 |
| Polymer MW (kDa) | 7.84 | 10.9 |
| Microsphere MW | 7.9 | 11.1 |
| (kDa) | | |

### Example 5-Preparation of Asenapine-Encapsulated PLA Polymer Microspheres-Batches 9-10

Following the general procedure described in Example 1, illustrated in FIG. 1, and detailed in Table 4, the DP was formed by dissolving either 4.50 g (Batch 9) or 2.25 (Batch 10) of acid-terminated Resomer® R 203 H (IV=0.29 dL/g) in either 18.90 g (Batch 9) or 9.47 g (Batch 10) of DCM and either 8.18 g (Batch 9) or 3.16 g (Batch 10) of BA (DCM/BA ratio=3:1 vol/vol)), followed by addition of either 5.63 g (Batch 9) or 3.87 g (Batch 10) of asenapine maleate with mixing until completely dissolved. The DP was filtered and pumped at a flow rate of 25 mL/min into a Levitronix® BPS-i100 integrated pump system operating at either 2,000 RPM (Batch 9) or 2,500 RPM (Batch 10). The CP comprising 0.35% PVA was also pumped into the homogenizer at a flow rate of 2 L/min (CP:DP=80:1).

The formed or forming microspheres exited the homogenizer and entered the SRV. Room temperature deionized water was added to the SRV (12 volume exchanges). Solvent removal was achieved using water washing (35-39° C.) and a hollow fiber filter. The bulk suspension was collected via filtration and lyophilized to obtain a free-flowing powder.

The process parameters and the characterization data for Batches 9-10 are shown in Table 4:

TABLE 4

| Batch # | 9 | 10 |
|---|---|---|
| Polymer | Resomer ® R 203 H | Resomer ® R 203 H |
| | acid-terminated | acid-terminated |
| Polymer IV | (IV = 0.29 dL/g) | (IV = 0.29 dL/g) |
| Solvent System | DCM/BA (3:1) | DCM/BA (3:1) |
| Surfactant | PVA 0.35% | PVA 0.35% |
| Homogenizer RPM | 2,000 | 2,500 |
| Total Yield (%) | 39 | 37 |
| Drug Load (%) | 17.4 | 19.0 |
| Encapsulation | 32.0 | 34.6 |
| Efficiency (%) | | |
| Residual Solvents | DCM/BA (ND/1.5) | DCM/BA (ND/2.2) |
| (% wt.) | | |
| Particle Size (D₁₀) | 6 | 8 |
| Particle Size (D₅₀) | 22 | 25 |
| Particle Size (D₉₀) | 41 | 47 |
| Polymer MW (kDa) | 25.1 | 23.9 |
| Microsphere MW | 25.3 | 25.1 |
| (kDa) | | |

FIG. 2 is a graph showing an amount of asenapine released in vitro over time from asenapine-encapsulated polymer microsphere Batches 9 and 10.

### Comparative Example 1-Preparation of Asenapine-Encapsulated PLGA Polymer Microspheres Batches 11-13

Following the general procedure described in Example 1, illustrated in FIG. 1, and detailed in Table 5, the DP was formed by dissolving either 2.25 g (Batch 11) of ester-terminated Viatel® DLG 8509 E (IV=0.82 dL/g), a poly (D,L-lactide-co-glycolide) (a “PLGA”) polymer having an 85:15 monomer ratio, or 4.50 g (Batch 12) of acid-terminated Viatel® DLG 8507 A (IV=0.62 dL/g), a PLGA polymer having an 85:15 monomer ratio, or 2.25 g (Batch 13) of ester-terminated Viatel® DL 02 E (IV=0.18 dL/g), a PLA polymer, in either 12.29 g (Batches 11 and 13) or 24.56 g (Batch 12) of DCM and either 4.09 g (Batches 11 and 13) or 8.19 g (Batch 12) of BA (DCM/BA ration=3:1 vol/vol)), followed by addition of either 3.87 g (Batches 11 and 13) or 7.75 g (Batch 12) of asenapine maleate with mixing until completely dissolved. The DP was filtered and pumped at a flow rate of 25 mL/min into a Levitronix® BPS-i100 integrated pump system operating at either 2,800 RPM (Batch 11) or 2,000 RPM (Batches 12 and 13). The CP comprising 0.35% PVA was also pumped into the homogenizer at a flow rate of 2 L/min (CP:DP=80:1).

The formed or forming microspheres exited the homogenizer and entered the SRV. Room temperature deionized water was added to the SRV (12 volume exchanges). Solvent removal was achieved using water washing (35-39° C.) and a hollow fiber filter. The bulk suspension was collected via filtration and lyophilized to obtain a free-flowing powder.

The process parameters and the characterization data for Batches 11-13 are shown in Table 5:

TABLE 5

| Batch # | 11 | 12 | 13 |
|---|---|---|---|
| Polymer | Viatel ® DLG 8509 E | Viatel ® DLG 8507 A | Viatel ® DL 02 E |
| | ester-terminated | acid-terminated | ester-terminated |
| | 85:15 | 85:15 | |
| Polymer IV | (IV = 0.82 dL/g) | (IV = 0.62 dL/g) | (IV = 0.18 dL/g) |
| Solvent System | DCM/BA (3:1) | DCM/BA (3:1) | DCM/BA (3:1) |
| Surfactant | PVA 0.35% | PVA 0.35% | PVA 0.35% |
| Homogenizer RPM | 2,800 | 2,000 | 2,000 |
| Total Yield (%) | 58 | 71 | 40 |
| Drug Load (%) | 31.2 | 34.6 | 16.5 |
| Encapsulation | 57 | 63 | 30 |
| Efficiency (%) | | | |
| Residual Solvents (% wt.) | DCM/BA (ND/3.6) | DCM/BA (ND/3.6) | DCM/BA (ND/0.8) |
| Particle Size (D₁₀) | 9 | 11 | 12 |
| Particle Size (D₅₀) | 29 | 33 | 27 |
| Particle Size (D₉₀) | 60 | 64 | 56 |
| Polymer MW (kDa) | 86.7 | 74.2 | 12 |
| Microsphere MW | 74 | 74 | 11.8 |
| (kDa) | | | |

FIG. 2 is a graph showing an amount of asenapine released in vitro over time from asenapine-encapsulated polymer microsphere Batches 11-13.

### Example 6-Pharmacokinetics Study in Rats of Batch Nos. 3, 4, 5, 6, and 9

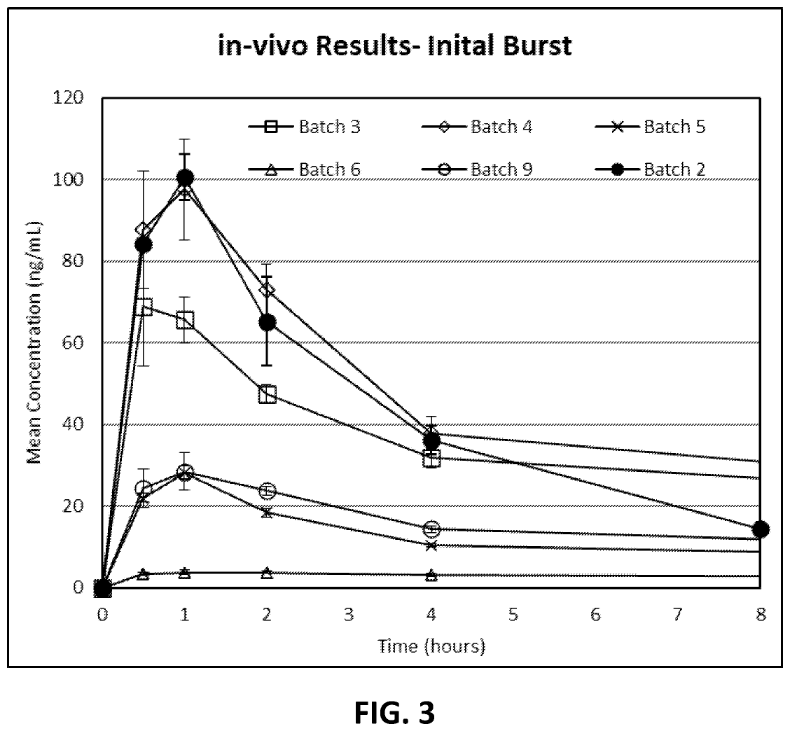

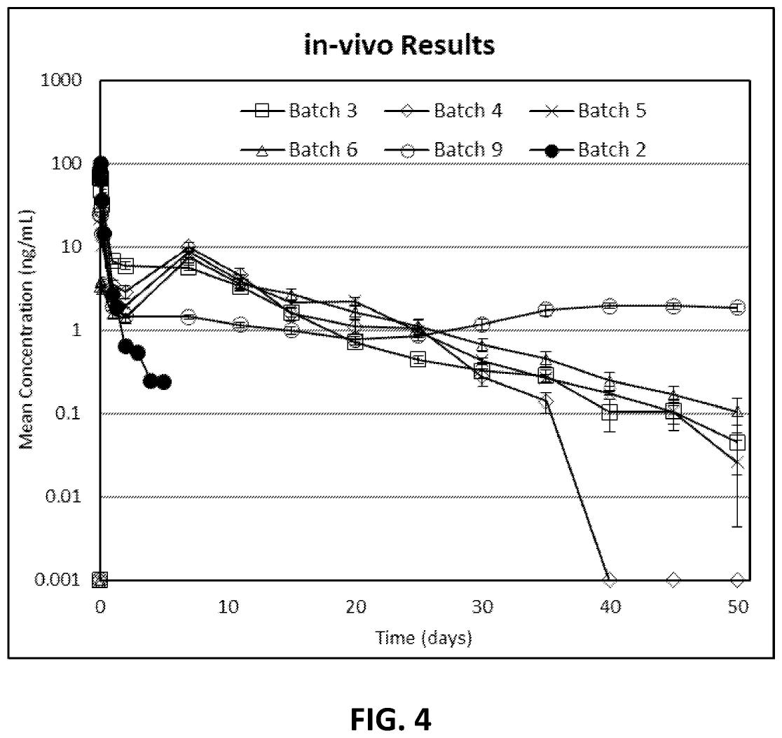

The pharmacokinetic profile of asenapine following a subcutaneously injected dose of time-released asenapine formulation in rats was studied. Five male rats per group received a 10 mg/kg dose of the indicated Batch No., having an asenapine concentration of 6.7 mg/mL (dose volume=1.5 mL/kg). Blood was collected pre-dose, at 0.5, 1, 2, 4, 24, and 48 hours, and at 7, 11, 15, 20, 25, 30, 35, 40, 45, and 50 days. FIGS. 3 and 4 are graphs showing the measured mean blood concentration (ng/ml) of asenapine as a function of time for Batches Nos. 3, 4, 5, 6, and 9.

In use, the microspheres may be suspended in a diluent for administration (injection). The diluent may generally contain a thickening agent, a tonicity agent, and a surfactant. The thickening agent may include carboxymethyl cellulose-sodium (CMC-Na) or other suitable compounds. An appropriate viscosity grade and suitable concentration of CMC-Na may be selected so that the viscosity of the diluent is 3 cps or higher. Generally, a viscosity of about 10 cps is suitable; however, a higher viscosity diluent may be preferred for larger microspheres in order to minimize the settling of microspheres in the suspension.

Uniform microsphere suspension without particle settling will result in a consistent delivered dose during drug administration by injection. To have a tonicity of the diluent closer to the biological system, about 290 milliosmole (mOsm), solutes such as mannitol, sodium chloride, or any other acceptable salt may be used.

The aspects disclosed herein are not intended to be exhaustive or to be limiting. A skilled artisan would acknowledge that other aspects or modifications to instant aspects can be made without departing from the spirit or scope of the invention. The aspects of the present disclosure, as generally described herein and illustrated in the figures, can be arranged, substituted, combined, separated, and designed in a wide variety of different configurations, all of which are contemplated herein.

Unless otherwise specified, “a,” “an,” “the,” “one or more of,” and “at least one” are used interchangeably. The singular forms “a”, “an,” and “the” are inclusive of their plural forms. The recitations of numerical ranges by endpoints include all numbers subsumed within that range (e.g., 1 to 5 includes 1, 1.5, 2, 2.75, 3, 3.80, 4, 5, etc.). The terms “comprising” and “including” are intended to be equivalent and open-ended. The phrase “consisting essentially of” means that the composition or method may include additional ingredients and/or steps, but only if the additional ingredients and/or steps do not materially alter the basic and novel characteristics of the claimed composition or method. The phrase “selected from the group consisting of” is meant to include mixtures of the listed group.

When reference is made to the term “each,” it is not meant to mean “each and every, without exception.” For example, if reference is made to microsphere formulation comprising polymer microspheres, and “each polymer microsphere” is said to have a particular API content, if there are 10 polymer microspheres, and two or more of the polymer microspheres have the particular API content, then that subset of two or more polymer microspheres is intended to meet the limitation.

The term “about” in conjunction with a number is simply shorthand and is intended to include+10% of the number. This is true whether “about” is modifying a stand-alone number or modifying a number at either or both ends of a range of numbers. In other words, “about 10” means from 9 to 11. Likewise, “about 10 to about 20” contemplates 9 to 22 and 11 to 18. In the absence of the term “about,” the exact number is intended. In other words, “10” means 10.

1. A microsphere formulation, comprising:
polymer microspheres, each polymer microsphere comprising:
(i) asenapine; and
(ii) a biodegradable polymer,
wherein each polymer microsphere comprises a drug load of asenapine of at least about 15% by weight of the polymer microsphere, and
wherein the polymer microspheres have a particle size of about 15 μm to about 40 μm (D₅₀).

2. The microsphere formulation of claim 1, wherein the asenapine comprises a salt.

3. The microsphere formulation of claim 1, wherein the biodegradable polymer comprises an acid terminated polylactide (PLA) polymer.

4. The microsphere formulation of claim 1, wherein the biodegradable polymer comprises an acid end-capped PLA polymer having an inherent viscosity (IV) of between about 0.1 dL/g and about 0.35 dL/g.

5. The microsphere formulation of claim 1, wherein the biodegradable polymer comprises an acid end-capped PLA polymer having an IV of between about 0.13 dL/g and about 0.29 dL/g.

6. The microsphere formulation of claim 1, wherein each polymer microsphere has an asenapine drug load of about 15 wt/wt % to about 25 wt/wt %.

7. The microsphere formulation of claim 1, wherein the polymer microspheres have a particle size of between about 15 μm about 25 μm (D₅₀).

8. A method for making a microsphere formulation, the method comprising: (A) mixing: (i) an acid end-capped polylactide (PLA) polymer having an inherent viscosity (IV) of between about 0.1 dL/g and 0.35 dL/g; (ii) dichloromethane; (iii) asenapine; and (iv) benzyl alcohol, to form a dispersed phase; (B) mixing: (i) water; and (ii) polyvinyl alcohol, to form a continuous phase; and (C) combining the dispersed phase with the continuous phase at a preselected flow rate ratio, dispersed phase: continuous phase, in a homogenizer.

9. The method of claim 8, wherein the acid end-capped PLA polymer has an IV of between about 0.13 dL/g and about 0.29 dL/g.

10. The method of claim 8, wherein the flow rate ratio dispersed phase: continuous phase is about 3:1.

11-13. (canceled)

14. A kit, the kit comprising polymer microspheres, each polymer microsphere comprising: (i) asenapine; and (ii) a biodegradable polymer comprising an acid end-capped polylactide (PLA) polymer, wherein each polymer microsphere comprises a drug load of asenapine of at least about 15% by weight of the polymer microsphere, and wherein the polymer microspheres have a particle size of about 15 μm to about 40 μm (D₅₀).

15. The kit of claim 14, further comprising a diluent for administration.

16. The kit of claim 14, wherein the asenapine comprises a salt.

17. The kit of claim 14, wherein the acid end-capped PLA polymer has an inherent viscosity (IV) of between about 0.1 dL/g and about 0.35 dL/g.

18. The kit of claim 14, wherein the acid end-capped PLA polymer has an IV of between about 0.13 dL/g and about 0.29 dL/g.

19. The kit of claim 14, wherein each polymer microsphere has an asenapine drug load of about 15 wt/wt % to about 25 wt/wt %.

20. The kit of claim 14, wherein the polymer microspheres have a particle size of between about 15 μm about 25 μm (D₅₀).
